(19) United States
(12) Patent Application Publication
(10) Pub. No.: US 2021/0322672 A1
(43) Pub. Date: Oct. 21, 2021

(54) Cradle Unit for Delivery Device

(71) Applicant: Debiotech S.A., Lausanne (CH)

(72) Inventor: Yannick Sublet, Lausanne (CH)

(21) Appl. No.: 17/269,016

(22) PCT Filed: Oct. 7, 2019
(86) PCT No.: PCT/IB2019/058527

§ 371 Date: Feb. 17, 2021

(30) Foreign Application Priority Data
Oct. 12, 2018 (EP) 18200265.9

(51) Int. Cl. A61M 5/14; A61M 5/142

(52) U.S. Cl. CPC A61M 5/1413; A61M 5/14248

(57) ABSTRACT

A delivery system comprises a delivery device and a cradle unit intended to be coupled to the delivery device. The cradle unit comprises patch device configured to be secured on the patient body (for example taped to the patient skin) and to receive the delivery device. The cradle unit may further comprise a transcutaneous connection device intended to provide a fluid communication between the delivery device and the patient body. The delivery system (for example at least one of the patch device and the delivery device) further comprises at least one locking device for locking the delivery device on the cradle unit and/or at least one alignment device configured to help the user to initiate/engage properly the delivery device on the cradle unit and/or at least one foolproof configured to prevent a bad coupling between the delivery device and the cradle unit.

### FIELD OF DISCLOSURE

The disclosure relates to a cradle unit intended to receive and/or to fix a delivery device on a patient.

### STATE OF THE ART

The new generations of delivery system are configured to be fixed on the patient body. These systems may comprise a delivery device and a cradle unit including a patch device and/or a transcutaneous connection device. The utilization of such assemblies enables a delivery device to be placed near the transcutaneous connection device by eliminating the kind of tubing between the delivery device and the transcutaneous connection device. Such systems allow the delivery device to be connected, disconnected and reconnected from the patch device when the patch device is placed on the patient body.

Placement of the delivery device on the patch device must be as easy as possible for the patient, and also must guarantee correct positioning in all cases.

Furthermore, the disconnection and/or reconnection of the delivery device from the patch device may be forbidden or unintended or unwanted or not required for some delivery device for several reasons (which may depend on the drug, the patient (baby, . . . ), . . . ).

### GENERAL DESCRIPTION OF THE DISCLOSURE

A first aspect of the disclosure is a delivery system configured to prevent a decoupling of a delivery device from its cradle unit when or as long as the cradle unit is fixed or placed on the patient body. According to this aspect of the disclosure, the delivery system comprises a delivery device and a cradle unit intended to be coupled to the delivery device. The cradle unit comprises patch device configured to be secured on the patient body (for example taped to the patient skin) and to receive the delivery device. The cradle unit may further comprise a transcutaneous connection device intended to provide a fluid communication between the delivery device and the patient body. The delivery system (for example at least one of the patch device and the delivery device) further comprises locking device for locking the delivery device on the patch device.

Preferentially, the locking device is configured to removably lock the delivery device on the patch device so as to allow disconnecting/decoupling the delivery device from the patch device if (and preferentially only if) the delivery system is not fixed on the patient body, for example when (after) the delivery system has been removed from the patient body or is no longer on the patient body.

Thus, once the delivery device has been locked on the patch device, the locking device may be de-activatable only if the patch device is no fixed on the patient body. Or once the delivery device has been locked on the patch device (and for example once the patch device has been fixed on the patient body), the locking device may be de-activatable after the patch device has been removed from the patient body.

Preferentially, the locking device is arranged in such a manner that the locking device is not reachable when the patch device is fixed/positioned on the patient body and the delivery device coupled to the patch device.

At least a part of the locking device may be arranged on a part of the cradle unit which is intended to be covered/overlaid by the delivery device when the delivery device is fixed on the cradle unit.

At least a part of the locking device may be arranged on the patch device which is intended to receive the delivery device.

At least a part of the locking device may be arranged on the patch device which is located between an top surface intended to receive the delivery device and a bottom surface intended to be fixed on the patient skin.

At least a part of the locking device may be arranged to be located between a lower of the delivery device and a bottom surface of the cradle unit intended to be fixed on the patient skin.

At least a part of the locking device may be arranged to face the patient skin when the delivery system is positioned/secured on the patient skin.

The at least a part of the locking device may comprise at least one of an operable portion, a hook member, an anchoring latch, a protrusion, a locking device, a notch, a groove and an opening.

The locking device may comprise a clip device (also called latch device) and an associated element intended to cooperate with clip device, for example when the delivery device is coupled with or locked to or fixed on the patch device. The clip device may be arranged on the patch device (resp. on the delivery device) and the associated element may be arranged on the delivery device (resp. on the patch device). The clip device may be a strip (for example with a latch) and the associated element may be an ergot. The clip device may be removed or moved by the user in order to unlock the locking device.

A second aspect of the disclosure disclosure is a delivery system configured to ensure correct positioning of the delivery device on the patch device. The delivery system comprises a delivery device and a cradle unit intended to be coupled to the delivery device. The cradle unit may comprise a patch device configured to be secured on the patient body (for example taped to the patient skin) and to receive the delivery device and an optional infusion set intended to be in fluid communication with the delivery device. The delivery system (at least one of the delivery device and the patch device) further comprises at least one alignment device for aligning the delivery device to the patch device. The at least one alignment device may include a sliding element and a guiding element configured to limit the motion of delivery device relative to the patch device according to at least one degree of freedom motion (or at least one dimension) when the sliding element slides against the guiding element.

Preferentially, the patch device comprises a first end and a second end defining a main axis of the patch device and the delivery device comprises an associated main axis defined by a third end and a fourth end. The infusion set may be arranged for example at/near/close to the first end and configured to be coupled to the third end. The delivery system may be configured to allow coupling of the delivery device with the patch device by a linear displacement of the delivery device relative to the patch device according to the main axis for example in order to ensure an operating/functional connection of the third end of the delivery device and the infusion set. The linear displacement is preferentially parallel to the surface of the patient skin.

Preferentially the third end may comprise an outlet port in fluid communication with a reservoir or a pumping device of the delivery device and the infusion set may comprise an inlet port (in fluid communication with a cannula or a needle intended to be inserted in the patient body) intended to be connected with the outlet port of the delivery device. In this case, an operating/functional connection may allow a fluidic communication (preferentially without leakage) between the reservoir or the pumping device of the delivery device with the infusion set.

The at least one alignment device may comprise a first alignment device configured to limit a yaw motion when the user couples the delivery device to the patch device. According to this embodiment, the guiding element of the first alignment device may extend on the patch device according the main axis until the first end of the patch device. And the sliding element of the first alignment device may extend on the delivery device according to the associated main axis from the third end of the delivery device. Both are configured to limit the yaw motion when the sliding element slides against the guiding element. The guiding element of the first alignment device may comprise a linear guide such as a hard ridge arranged on the patch device and the sliding device of the first alignment device may comprise a groove arranged on the delivery device (for example a groove arranged on the housing of the delivery device).

The at least one alignment device may (further) comprise a second alignment device configured to limit at least one of a pitch motion, a roll motion and a perpendicular motion (to the main axis) when the user couples the delivery device to the patch device. According to this embodiment, the sliding element of the second alignment device may extend on the patch device according the main axis and the guiding element of the second alignment device may extend on the delivery device according to the associated main axis. Both are configured to limit at least one of the pitch motion, the roll motion and/or optionally a perpendicular motion relative to the main axis when the guiding element of the second alignment device slides against the sliding element of the second alignment device.

Furthermore, the sliding element of the second alignment device may further comprise a protrusion configured to prevent the coupling between the delivery device and the infusion set if the guiding element of the second alignment device and the sliding element of the second alignment device are not coupled in a correct manner.

In some embodiments, the delivery system further comprises a final alignment device comprising a first part arranged on the delivery device and a second part arranged on the infusion set. The final alignment device may be configured to prevent the final coupling between the delivery device and the infusion set if the delivery device is incorrectly engaged on the cradle unit.

In some embodiments, the delivery system further comprises at least one foolproof configured to prevent the coupling between the delivery device and the cradle unit if the delivery device is not properly engaged on the cradle unit.

In some embodiments, the delivery device described in the first aspect of the disclosure may further comprise the features of the delivery device described in the second aspect of the disclosure and/or vice versa.

### LIST OF FIGURES

The present disclosure will be better understood at the light of the following detailed description which contains non-limiting examples illustrated by the following figures:

FIG. 1 shows degrees of freedom motion according to the present document.

FIG. 2 shows an example of a delivery system.

FIG. 3 shows a 3d view of an example of a cradle unit.

FIG. 4 shows a bottom view of an example of a delivery system.

FIG. 5 shows a bottom view of an example of a delivery device.

FIGS. 6a, 6b and 6c show sequential views of coupling between a transcutaneous connection device and a delivery device

FIGS. 7a and 7b show schematic views of two coupling.

FIGS. 8a, 8b, and 8c show views of three distinct coupling.

FIGS. 9a, 9b, 9c and 9d show sequential views of coupling between a delivery device and a cradle unit.

FIG. 10 shows an example of a delivery system.

### LIST OF ELEMENTS

1 yaw motion
2 roll motion
3 pitch motion
4 Z axis
5 Y axis
6 X axis
10 delivery system
11 delivery device
12 cradle unit
13 housing
14, 14a, 14b linear guide/guiding element
15 locking member
15′ hook member/anchoring latch
15″ operatable portion
16 needle
17 protective element
18 patch device
19 transcutaneous connection device
20 ridge/guiding element
21, 21a, 21b slide/sliding element
22 end of guiding element 23 locking member 24 hook member/anchoring latch 25 operatable portion 26 opening 27 adhesive layer 28 main axis 29 associated main axis 30 housing 31 first end 32 second end 33 third end 34 fourth end 35 needle or cannula 36 septum 37 internal channel 38 stress of the septum 39 leakage 40 corresponding female member (of the locking element)/notch/groove 41 protrusion 42 flexible/weak/soft portion 43 corresponding member (of the locking device)/notch/groove 45 slot/groove/sliding element 46 opened end 47 bottom surface 48 mechanical stop 49 foolproof element 100 Delivery system 101 Patient skin 102 Delivery device 103 Cradle unit 104 Adhesive layer 105 Drug reservoir 106 Pumping device 107 Energy source 108 Coupling device

### DETAILED DESCRIPTION OF THE DISCLOSURE

In the following detailed description, reference is made to the accompanying drawings that form a part hereof, and in which are shown by way of illustration several embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise.

As used in this specification and the appended claims, any direction referred to herein, such as “top”, “bottom”, “left”, “right”, “upper”, “lower”, and other directions or orientations are described herein for clarity in reference to the figures and are not intended to be limiting of an actual device or system. Devices and systems described herein may be used in a number of directions and orientations.

As used herein, “have”, “having”, “include”, “including”, “comprise”, “comprising” or the like are used in their open ended sense, and generally mean “including, but not limited to.

As used herein, “at least one of A, B, and C”, “at least one of A, B or C”, “selected from the group consisting of A, B, C, and combinations thereof” or the like are used in their open ended sense including “only A, or only B, or only C, or any combination of A, B and C” unless the content clearly dictates otherwise.

As used herein, the term “or” is generally employed in its sense including “and/or” unless the content clearly dictates otherwise.

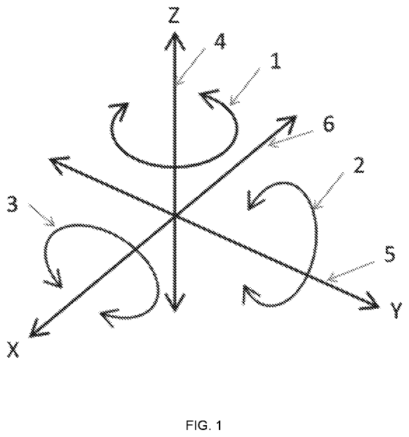

As used herein, “degree of freedom motion” means, as shown in FIG. 1, a path 1, 2, 3, 4, 5 or 6. A degree of freedom motion may be translational, such as along axes 4, 5 or 6 or may be rotational, such as along arcs 1, 2 or 3.

When the delivery device is moved (by the user) (relative to the patch device), the displacement may comprise translational movements (intentional or not), for instance, translation along a lateral direction (6), a longitudinal direction (5), an elevation direction (4) or a combination of lateral (6), longitudinal (5) and elevation (4) directions. Furthermore, the delivery device may be pivoted (intentional or not) along a path may include a yaw motion (1), a pitch motion (3), a roll motion (2) or a combination of yaw motion (1), pitch motion (3) and roll motion (2).

The sliding element and the guiding element may be configured to prevent at least one of a lateral motion, an elevation motion, a yaw motion, a pitch motion and a roll motion when the user couples the delivery device to the patch device.

### Description of the System

According to some embodiments as shown by the FIG. 1, the delivery system (10) may comprise a delivery device (11) and a cradle unit (12).

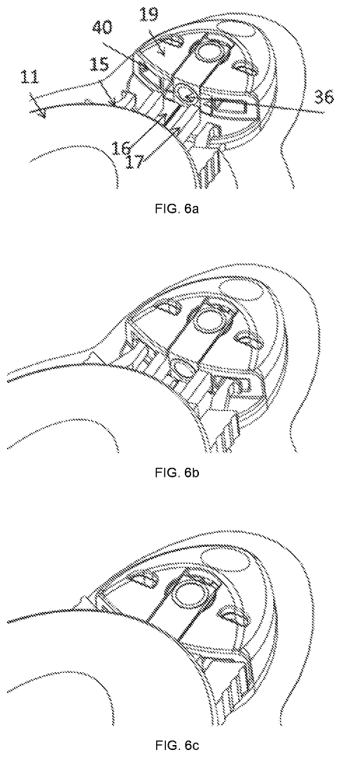

The delivery device (11) may comprise at least one of:
an housing (13),
a reservoir (not shown) configured to store a solution to be infused to a patient which may be arranged into the housing,
a fluidic pathway (not shown) in/through which the solution may flow, which may comprise an outlet port which may extend away from the housing for example a needle (16),
A pumping device (not shown) configured to move the solution from the reservoir to the patient through the fluidic pathway, which may be arranged into the housing,
One or more guiding element (14),
One or more sliding element (45) (shown in the FIG. 5),
Protective element (17),
A locking member (15), and
Optionally, an electronic device (not shown) which may control and/or monitor the pumping device and may be arranged into the housing.

The cradle unit (12) may comprise at least one of:
A patch device (18) configured to receive the delivery device,
A transcutaneous connection device (19) (also called infusion set) which may comprise a housing (30), an inlet port configured to be connected to the outlet port of the delivery device so to allow a fluidic communication of the delivery device (in an operating position) to the patient body,
One or more guiding element (20),
One or more sliding element (21a, 21b), and
One or more locking member (23).

The cradle unit (12) may be made in a single piece comprising the patch device and the transcutaneous connection device (19). The cradle may comprise two distinct pieces, a first piece which may comprise the patch device and a second piece which may comprise the transcutaneous connection device (19). In this case, the transcutaneous connection device may be placed on the patient body and after the patch device (18) may be coupled to the transcutaneous connection device in order to form the cradle unit (12). In another embodiment, the patch device may be placed first and then the transcutaneous connection device may be coupled to the patch device.

The cradle unit (12) and/or the patch device (18) may comprise a first end (31) and a second end (32) disposed opposite to the first end. The first end and the second end may define a main axis (28) of the cradle unit or of the patch device (18). Preferentially, the transcutaneous connection device (19) is arranged at/near/close to the first end.

The delivery device (11) may comprise a third end (33) and a fourth end (34) disposed opposite to the third end. The third end and the fourth end may define an associated main axis (29) of the delivery device (11). The third end (for example the outlet port) may be configured to cooperate with at least one of the first end and the transcutaneous connection device (for example the inlet port).

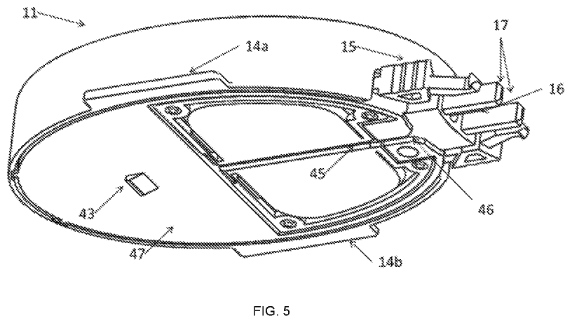

The transcutaneous connection device (19) may comprise at least one of a housing (30), an internal channel (37) extending from the inlet port to a needle or cannula (35) intended to be inserted into the patient body (as shown by the FIG. 4). The inlet port may comprise a septum (36) configured to seal the inlet port and to be pierced by the needle (16) of the delivery device (11). Or inversely, the inlet port may comprise a needle configured to pierce a septum of the outlet port of the delivery device.

The cradle unit may comprise an adhesive layer (27) arranged at least a part on a bottom surface of the cradle unit (12). The adhesive layer may extend away from an edge of the cradle unit.

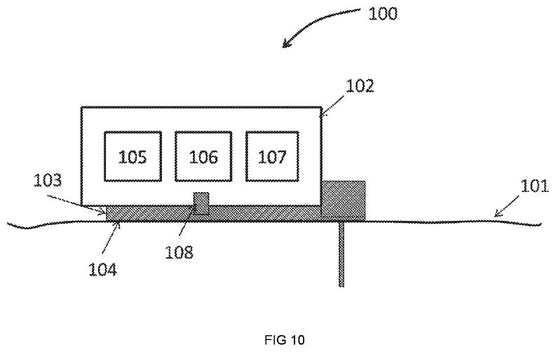

The FIG. 10 discloses a similar embodiment arranged on the patient skin (101). The delivery system (100) comprises a delivery device (102) and a cradle unit (103) on which the delivery device (102) can be preferentially rigidly fixed (optionally removably). The cradle unit may comprise a bottom surface comprising an adhesive layer (104) configured to contact the patient skin (101). The surface of the adhesive layer (104) may be greater than the bottom surface on at least one dimension in order to improve the adherence of the system on the patient skin. The cradle unit may further comprise an top surface configured to receive the delivery device (102). The top surface receiving the delivery device may be smaller than a bottom surface of the delivery device in order to improve the comfort of the patient. In other embodiment, the top surface receiving the delivery device may have substantially the same shape and size as the bottom surface of the delivery device.

The delivery device (102) may comprise a housing in which is arranged (at least partially) at least one of a drug reservoir (105), a pumping device (106), and energy source (107) for the pumping device. In one embodiment, the energy source is a battery and the pumping device is a displacement pump. In another embodiment, the energy source is a propellant and the pumping device is an element which compresses the drug reservoir.

Preferentially, the delivery system comprise at least one coupling device (108) (as described in this document for example a lock device and/or a guiding device) configured to rigidly (optionally removably) fix/secure the delivery device to the cradle unit. More preferentially, the coupling device fixes the housing (in which the drug reservoir is arranged) of the delivery device to the cradle unit.

### Features of a Locking Device of the System

The delivery system may comprise one or more locking device which may be configured to lock (optionally removably lock) the delivery device (11) to the cradle unit (12).

The locking device may comprise a locking member and a corresponding member configured to mechanically cooperate with the locking member to lock the coupling.

A first locking device may be configured to lock the delivery device to the cradle unit, for example to the patch device. The patch device may comprise a locking member and the delivery device may comprise a corresponding member (such as a groove or slot or opening or a protrusion). The locking member may comprise a hook member and an operatable portion which may be configured to be moved (pulled or pushed or removed or broken) so as to move the hook member relative to the delivery device and/or the cradle unit in order to lock and/or unlock for example by an action of the user.

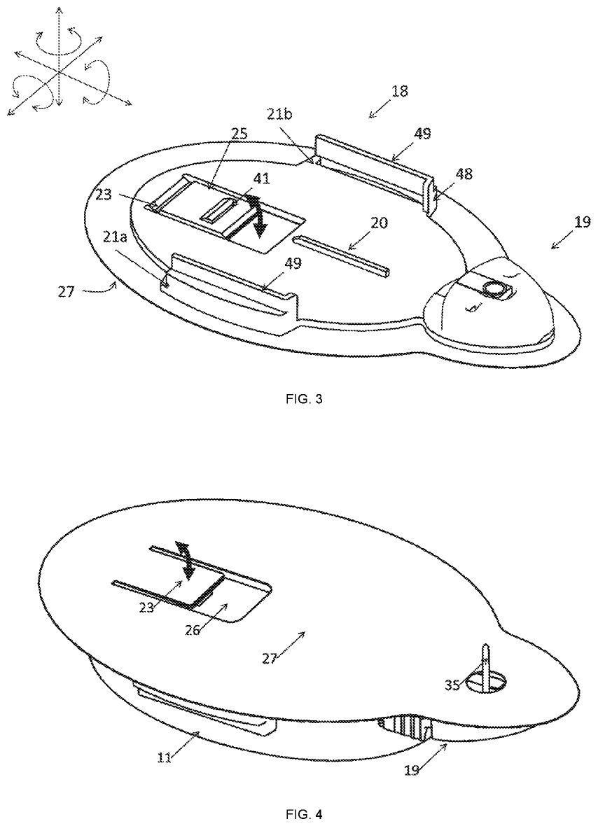

The FIG. 3 shows more details of an example of a locking member. The hook member (24) may comprise a protrusion (41) which extends from an top surface of the patch device (for example an top surface of the locking member, more particularly of the operatable portion). The protrusion may comprise a beveled corner which cooperates with the delivery device in order to facilitate the displacement of the operatable portion during the coupling process (when the delivery device is placed (slid) on the patch device). The locking member (23) may comprise a flexible/weak/soft portion configured to bend or to break by the user (for example by a pulling action on the operatable portion).

The FIG. 5 shows more details of the corresponding member (43) of the delivery device (11). The corresponding member (43) may be arranged on a bottom surface (47) of the delivery device and configured to cooperate with the locking member of the patch device during the coupling process, more preferentially to lock the coupling when the delivery device (11) is substantially fully coupled to the cradle unit. The corresponding member (43) may comprise a protrusion which may have a beveled corner. In other potential embodiment, the corresponding member may comprise a slot, groove or opening arranged in/through a bottom surface of the delivery device.

The FIG. 4 shows a bottom view of the delivery system (10). Once the cradle unit has been placed on the patient body and the delivery device (11) has been placed (locked or coupled) on the patch device (18), the operatable portion cannot be reached (it is no longer accessible). For example, this operatable portion may be arranged between the bottom surface of the delivery device and the patient skin (or a bottom surface of the cardle unit). Thus, the delivery device may cover the operatable portion when the delivery device is placed on the cradle unit.

The cradle unit (12) or the patch device (18) may comprise an opening (26) configured to allow reaching the operatable portion (25) of the locking member (23) when the delivery system is not fixed/placed on the patient body, for example once the delivery system was removed from the patient skin. Preferentially at least a part of the opening (26) (and/or the operaratble portion) does not comprise any adhesive layer in order to reach an end of the locking member (23) when the delivery device (11) is fixed/coupled on the patch. Thus, to unlock this locking device, the user has to remove the delivery system (in more particularly the cradle unit on which the delivery device is placed) from the patient body. In other terms, to remove the delivery device (11) from the cradle unit or the patch device, the user (for example the patient or a nurse or a caregiver) has to remove the overall system. This feature is important to ensure that the patient cannot remove (and optionally reconnect) the delivery device from the cradle unit during the whole period of treatment. Thus, the delivery device (11) cannot be removed from the cradle unit (12) as long as the delivery device (11) and the cradle unit (12) are placed and/or lock on the patient skin.

Thus, to receive the treatment, a user has to fix the cradle unit on the patient body (and optionally insert the needle or cannula through the patient body). Once the cradle unit fixed on the patient body (and the needle/cannula inserted), the user has to couple the delivery device on the cradle unit. To couple the delivery device to the cradle unit, the user may carry out a translational motion (for example parallel to the patient skin surface on which the cradle is fixed) of the delivery device relative to the patch device until the locking device locks (for example until a sound “clip”). At the end of the treatment (only at the end of the treatment) (for example when the reservoir is empty or after a predetermined time period) or in case of failure of the delivery system, the delivery system can be removed in one piece from the patient body. Once the overall system removed, the user may unlock the delivery device from the patch device/cradle unit.

In one embodiment, the transcutaneous has to be placed with a specific tool (for example an inserter as disclosed by the application WO2013/068900, the contents of which are incorporated by reference in the present document). Thus, when the caregiver/user removes the system from the patient, the caregiver/user knows that the patient has kept the delivery device on the cradle unit (at least) until the delivery system has been removed from the patient body; For example during the whole period of treatment. It is an important feature for example when the delivery device is configured to automatically deliver a drug during or after a predetermined time period and that the drug is expensive.

Preferentially, the cradle unit (12) is configured to be discarded after a single use. For example, once the cradle unit removed from the patient, the cradle cannot be reused (a tool may be required to place the cradle unit (for example to insert the cannula)). This feature may be useful to prevent any contamination (between two patients) and to ensure that the patient received the whole treatment.

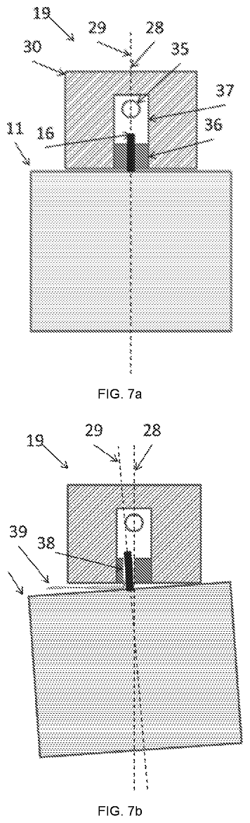

According to some embodiments as disclosed by the FIGS. 6a, 6b and 6c, a second locking device may be configured to lock the third end (of the delivery device) to the first end (of the cradle unit), for example a portion comprising the outlet port of the delivery device (11) to the transcutaneous connection device (19). The delivery device (11) may comprise a locking member (15) and the transcutaneous connection device may comprise a corresponding (female) member (40). The locking member (15) may comprise a hook member (15′) and an operatable portion (15″) which may be configured to be moved (pushed or pulled) so as to move the hook member (15′) relative to the delivery device and/or the cradle unit in order to lock and/or unlock by a press action of the user on the operatable portion. The FIGS. 6a, 6b and 6c show a focus view on the locking device during the coupling process.

If the delivery device comprises the first locking device and the second locking device, to remove the delivery device from the cradle unit, the user has to unlock the first locking device (for example bend or break the locking member of the first locking device) and then unlock the second locking device or vice versa.

### Features of an Alignment Device of the System

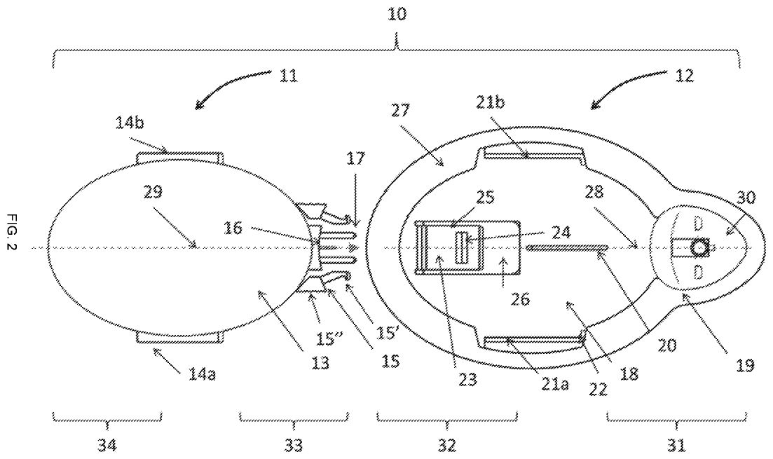

As explained above, preferentially the cradle unit may be placed first on the patient body and after the delivery device may be placed on the cradle unit. The cradle unit may be fixed on the patient skin surface by an adhesive layer disposed on its bottom surface. Once the cradle unit fixed on the patient skin, the cradle unit defines a spatial reference (for example in Euclidean or geometric space) which has to be taken into account for coupling the delivery device on the cradle unit (or the patch device). The FIGS. 1 and 2 define the degrees of freedom motion of the delivery device (11) in a reference space defined by the cradle unit (or patch device).

Preferentially, the delivery system (10) comprises one or more alignment device which may be configured to allow an alignment of the delivery device with the cradle unit (patch device and/or transcutaneous connection device). This feature may be important because during the coupling (or assembly) process (of the delivery device to the cradle unit) the associated main axis (29) (of the delivery device) has to be (perfectly) parallel to the main axis (28) (of the patch device or cradle unit) so as not to stress the coupling between the outlet port of the delivery device (which may comprise a needle or a septum) and the inlet port of the transcutaneous connection device (19) (which may comprise a septum or a needle)

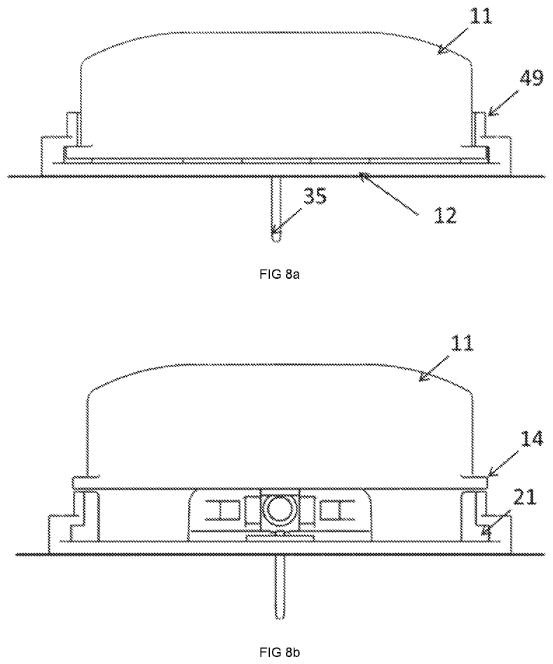

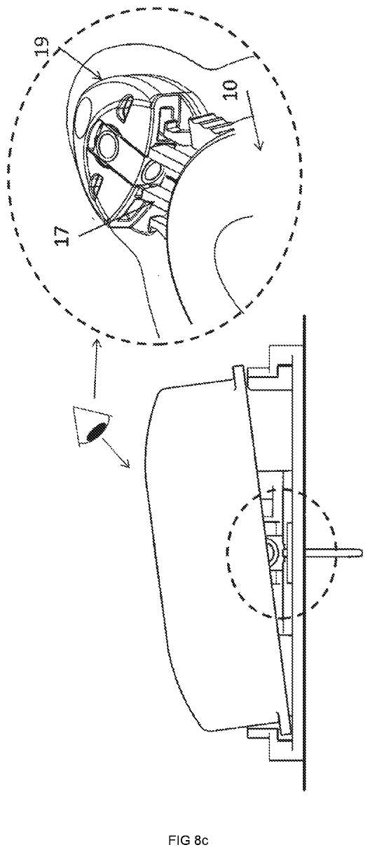

If the delivery device is not correctly positioned over (aligned to) the cradle unit, then the delivery device cannot be used. Indeed, in this case, either the delivery device cannot be fixed (locked) to the patch device (for example prevented by at least one foolproof or a final alignment device such as a protective element (17) (see FIGS. 8b and 8c) or the septum is pierced obliquely by the needle (see FIG. 7b).

The FIG. 7a shows a schematic sectional view of the delivery device (11) and the transcutaneous connection device (19). Both axes (28, 29) are parallel and the septum correctly seals the internal channel around the needle. The FIG. 7b shows another schematic sectional view of the delivery device (11) and the transcutaneous connection device (19). In this case, the main axis (28) and the associated main axis (29) is not parallel and the needle induce a stress (38) to the septum, a leakage (39) may occur.

The delivery system may comprise one or more alignment device so as to limit at least one degree of freedom motion of the delivery device during the coupling process. The alignment device, the sliding element and guiding element disclosed thereafter are presented to provide examples, the following detailed description contains non-limiting embodiments illustrated by the figures.

The delivery system may comprise a first alignment device configured to limit a yaw motion of the delivery device relative to the cradle unit (or patch device). The first alignment device may comprise a guiding element and a sliding element configured to slide against the guiding element.

According to some embodiments as shown by the FIG. 3, the guiding element of the first alignment device may comprise one or more ridge (20) which may be arranged on the patch device (18) (or on the delivery device). The ridge (20) is preferentially a hard ridge. The ridge (20) may extend, in whole or in part, along the main axis (28). Preferentially, the ridge (20) extends in whole or in part of the first end. The ridge (20) may be arranged in the center of the patch device (or of the delivery device). Preferentially, in order to facilitate the insertion of the guiding element (20) into the sliding element (45), the guiding element/ridge (20) may comprise at least one bevelled angle.

According to some embodiments as shown by the FIG. 5, the sliding element of the first alignment device may comprise a slot/groove (45) which may be arranged in the delivery device (11) (or on the patch device). The slot/groove (45) is preferentially arranged at a bottom surface (47) of the delivery device (11) (or of the patch device). The slot/groove (45) may extend, in whole or in part, along the associated main axis (29). Preferentially, the slot/groove (45) extends in whole or in part of the third end. The slot/groove (45) may be arranged in the center of the delivery device (or of the patch device). Preferentially, in order to facilitate the insertion of the guiding element (20) into the sliding element (45), the sliding element (45) may comprise an opened end.

The delivery system may comprise a second alignment device configured to limit at least one of a pitch motion, a roll motion and a perpendicular motion of the delivery device relative to the cradle unit (or patch device). The perpendicular motion may be relative to the main axis, for example a translation along a lateral direction, an elevation direction or a combination of lateral and elevation directions. The second alignment device may comprise a guiding element and a sliding element configured to slide against the guiding element.

According to some embodiments as shown by the FIG. 3, the sliding element of the second alignment device may comprise one or more slide (21a, 21b) which may be arranged on the patch device (18) (or on the delivery device). The sliding element may comprise a concave cavity configured to receive a linear guide or guiding element which may have a convex shape. The sliding element may extend, in whole or in part, along the main axis (28). Preferentially, a first sliding element (21a) may be arranged on a first edge of the patch device (or of the delivery device) and a second sliding element (21b) may be arranged on a second edge of the patch device (or of the delivery device). The second edge may be opposite to the first edge. The first sliding element (21a) may be disposed substantially parallel to the second sliding element (21b). Preferentially, in order to facilitate the insertion of the guiding element (14) into the sliding element (21a, 21b), the sliding element (21a, 21b) may comprise an opened end.

According to some embodiments as shown by the FIG. 5, the guiding element of the second alignment device may comprise a linear guide (14a, 14b) (such as a ridge or protrusion) which may be arranged on the delivery device (11) (or on the patch device). Preferentially, a first guiding element (14a) may be arranged on a first edge of the delivery device (or of the patch unit) and a second guiding element (14b) may be arranged on a second edge of the delivery device (or of the patch unit). The second edge may be opposite to the first edge. The first guiding element (14a) may be disposed substantially parallel to the second guiding element (14b). The guiding element (14a, 14b) may extend, in whole or in part, along the associated main axis (29). Preferentially, in order to facilitate the insertion of the guiding element (14) into the sliding element (21), the guiding element (14) may comprise at least one beveled angle.

Preferentially, the alignment device may comprise at least one foolproof element. The foolproof element is configured to prevent a connection of the delivery device with the cradle unit or transcutaneous connection device when the delivery device is not correctly aligned with the cradle unit. The foolproof element may be configured to overstate the alignment error. According to some embodiments as shown by the FIGS. 3, 8a, 8b, and 8c, the sliding element (21) of the second alignment device may further comprise a foolproof element (49). The foolproof element (49) may comprise a protrusion arranged on sliding element (21), which may extend in an elevation direction (4). The FIGS. 8a, 8b, and 8c show three distinct positions of the delivery device (11) relatively to the cradle unit (12). In the FIG. 8a, the delivery device (11) is correctly aligned with the cradle unit (12). In the FIGS. 8b and 8c, the delivery device (11) is not correctly aligned with the cradle unit (12).

The first alignment device and the second alignment device may be configured in order to allow a translation along the longitudinal direction (5) which is parallel to the main axis (28) until the final coupling for example a mechanical stop.

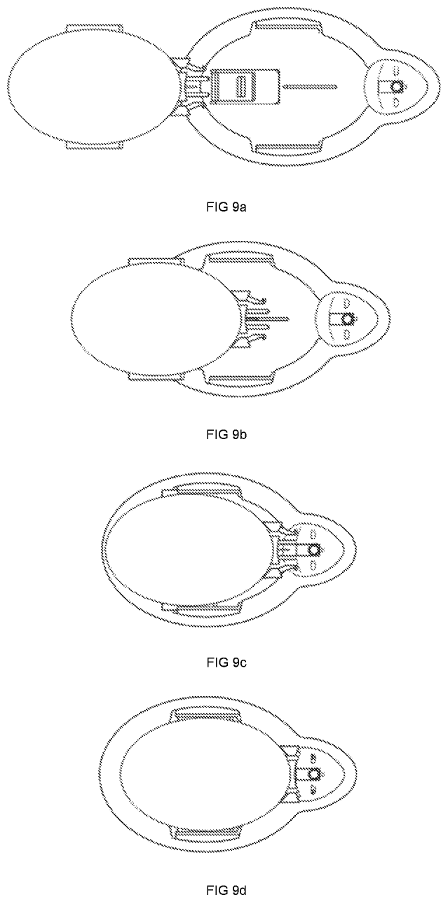

The FIGS. 9a-d illustrates the coupling process.

The protective element (17) of the delivery device (11) may be configured to protect the needle (16) to any contact from an external element or to cooperate with a part of the cradle unit (for example a transcutaneous connection device) and used as an alignment device. The protective element (17) extends from the delivery device and may be longer than the needle. The protective element (17) may comprise one or more cantilever beam. Preferentially, the protective element comprises two cantilever beams arranged on each side of the needle but spaced from the needle (16). The protective element (17) may have at least one of rectangular shaped section, ovoid shaped section, ring shaped section, closed curve shaped section and other polygonal shaped section. The transcutaneous connection device (12) may comprise a cooperative element (for example cooperating with the at least one feature or shape of the protective element) having a shape configured to allow or not the coupling (for example the piercing of the septum) and/or to carry out the final alignment.

As shown by FIG. 8c, the protective element (17) may be considered as a final alignment device or a foolproof. Such final alignment device may be arranged at an end of the delivery device (10) (for example the third end as disclosed by the FIG. 2) and may be configured to be mechanically coupled to the transcutaneous connection device or to the cradle unit in a vicinity of the transcutaneous connection device. The final alignment device (or foolproof) may be configured to mechanically cooperate with the transcutaneous connection device, for example to be inserted into opening (having substantially the same shape of the final alignment device) of the transcutaneous connection device.

In one embodiment, the outlet port of the delivery device may comprise a pierceable septum and the transcutaneous connection device may comprise a needle configured to pierce the septum of the delivery device in order to provide a fluidic connection.

1-23 (canceled)

24. A delivery system comprising:
a delivery device;
a cradle unit configured to be coupled to the delivery device, the cradle unit including a patch device configured to be fixed on a body of a patient and to receive the delivery device; and
a locking device configured to lock the delivery device on the patch device while the delivery system is fixed on the body of the patient.

25. The delivery system according to claim 24, wherein when the delivery device is locked on the patch device, the locking device is unlockable only when the patch device is not fixed on the body of the patient.

26. The delivery system according to claim 24, wherein when the delivery device is locked on the patch device, the locking device is unlockable after the patch device has been removed from the body of the patient.

27. The delivery system according to claim 24, wherein the locking device is configured to be unreachable when the patch device is fixed on the body of the patient and when the delivery device coupled to the patch device.

28. The delivery system according to claim 24, wherein at least a part of the locking device is arranged on a part of the cradle unit which is configured to be covered by the delivery device when the delivery device is fixed to the cradle unit.

29. The delivery system according to claim 24, wherein at least a part of the locking device is arranged on the patch device that is configured to receive the delivery device.

30. The delivery system according to claim 24, wherein at least a part of the locking device is arranged on the patch device which is located between a top surface configured to receive the delivery device and a bottom surface configured to be fixed to a skin of the patient.

31. The delivery system according to claim 24, wherein at least a part of the locking device is arranged between a lower surface of the delivery device and a bottom surface of the cradle unit configured to be fixed to a skin of the patient.

32. The delivery system according to claim 24, wherein at least a part of the locking device is configured to face a skin of the patient when the delivery system is fixed to the skin of the patient.

33. The delivery system according to claim 24, wherein the locking device includes a clip device and an associated element.

34. The delivery system according to claim 33, wherein the clip device is arranged on the patch device and configured to be covered by the delivery device when the delivery device is coupled to the patch device.

35. The delivery system according to claim 33, wherein the associated element is arranged on the delivery device.

36. A delivery system comprising:
a delivery device; and
a cradle unit configured to be coupled to the delivery device in a determined position, the cradle unit including,
a patch device configured to be secured on a body of a patient and configured to receive the delivery device, and
an infusion set configured to be in fluid communication with the delivery device,
an alignment mechanism for aligning the delivery device to the patch device, the alignment mechanism including,
a sliding element and a guiding element configured to limit a motion of delivery device relative to the patch device according to a degree of freedom when the sliding element slides against the guiding element, configured to ensure an operating connection between the delivery device and the infusion set at an end of a displacement performed by the sliding.

37. The delivery system according to claim 36, wherein the patch device comprises a first end and a second end defining a main axis of the patch device and the delivery device comprises an associated main axis defined by a third end and a fourth end, wherein the infusion set is arranged at the first end and configured to be coupled to the third end.

38. The delivery system according to claim 37, wherein the delivery system is configured to couple to the delivery device with the patch device by a linear displacement of the delivery device relative to the patch device along the main axis.

39. The delivery system according to claim 36, wherein the alignment mechanism comprises a first alignment device configured to limit a yaw motion when the user couples the delivery device to the patch device.

40. The delivery system according to claim 39, wherein the guiding element of the first alignment device extends on the patch device according the main axis to the first end of the patch device and the sliding element of the first alignment device extends on the delivery device according to the associated main axis from the third end of the delivery device in such a manner to limit the yaw motion when the sliding element slides against the guiding element.

41. The delivery system according to claim 36, wherein the alignment mechanism comprises a second alignment device configured to limit at least one of a pitch motion and a roll motion when the user couples the delivery device to the patch device.

42. The delivery system according to claim 41, wherein the sliding element of the second alignment device extends on the patch device according the main axis and the guiding element of the second alignment device extends on the delivery device according to the associated main axis in such a manner to limit at least one of the pitch motion, the roll motion and an perpendicular motion relative to the main axis when the guiding element of the second alignment device slides against the sliding element of the second alignment device.

43. The delivery system according to claim 41, wherein the sliding element of the second alignment device further comprises a protrusion configured to prevent the coupling between the delivery device and the infusion set if the guiding element of the second alignment device and the sliding element of the second alignment device are not coupled in a correct manner.

44. The delivery system according to claim 36, further comprising:
a final alignment device having a first part arranged on the delivery device and a second part arranged on the infusion set.

45. The delivery system according to claim 44, wherein the final alignment device is configured to prevent the final coupling between the delivery device and the infusion set when the delivery device is incorrectly engaged on the cradle unit.

46. The delivery system according to claim 36 further comprising:
a foolproof configured to prevent the coupling between the delivery device and the cradle unit when the delivery device is not properly engaged on the cradle unit.
